(19) United States
(12) Patent Application Publication
(10) Pub. No.: US 2006/0099189 A1
(43) Pub. Date: May 11, 2006

(54) Anti-cancer virus desensitization method

(76) Inventor: Robert M. Lorence, Bethesda, MD (US)

Correspondence Address: LEWIS J. KREISLER, LEGAL DEPARTMENT, 930 CLOPPER ROAD, GAITHERSBURG, MD, 20878, US

(21) Appl. No.: 10/547,654

(22) PCT Filed: Mar. 2, 2004
(86) PCT No.: PCT/US04/06158

§ 371 Date: Sep. 1, 2005

Related U.S. Application Data
(60) Provisional application No. 60/457,035, filed on Mar. 24, 2003.

(51) Int. Cl. A61K 48/00; A61K 35/76

(52) U.S. Cl. 424/93.6

(57) ABSTRACT

A mammalian subject having a tumor is treated by a method comprising administering to the subject an amount of a Newcastle disease virus effective to treat the subject, wherein the virus is administered to the subject in one or more cycles; at least one cycle comprises administering sequentially one or more desensitization doses of the followed by one or more escalated doses of the virus to the subject; the amount of the virus in each escalated dose is higher than the amount of virus in each desensitization dose; and the first escalated dose is administered from 18 to 36 hours after the first desensitization dose.

### BACKGROUND OF THE INVENTION

The administration of a desensitizing dose of an oncolytic virus before higher subsequent doses is disclosed in WO 00/62735 (pages 35-36). See also Pecora, et al., J. Clin. Oncol. (May 2002) 20(9):2251-2266; and Bergsland, et al., J. Clin. Oncol. (May 2002) 20(9): 2220-2222.

The administration of oncolytic viruses using an intravenous pump, syringe pump, intravenous drip or slow injection over the course of 4 minutes to 24 hours, for example over the course of 20 to 60 minutes, is disclosed in WO 00/62735 (page 36, lines 16-19).

### SUMMARY OF THE INVENTION

This invention provides a method for treating a mammalian subject having a tumor, comprising administering to the subject an amount of a Newcastle disease virus effective to treat the subject, wherein the virus is administered to the subject in one or more cycles; at least one cycle comprises administering sequentially one or more desensitization doses of the followed by one or more escalated doses of the virus to the subject; the amount of the virus in each escalated dose is higher than the amount of virus in each desensitization dose; and the first escalated dose is administered from 18 to 36 hours after the first desensitization dose.

This invention is based on the finding that desensitization to Newcastle Disease Virus occurs in a short time (e.g. 24 hours) after the desensitizing dose.

### DETAILED DESCRIPTION OF THE INVENTION

As used herein the transitional term “comprising” is open-ended. A claim utilizing this term can contain elements in addition to those recited in such claim. Thus, for example, the claims can read on treatment regimens that also include other therapeutic agents or therapeutic virus doses not specifically recited therein, as long as the recited elements or their equivalent are present.

As used herein “NDV” is an abbreviation for Newcastle Disease Virus. As used herein “DLT” is an abbreviation for dose limiting toxicity. As used herein the term “plaque-forming unit” (PFU) means one infectious virus particle. As used herein “BPFU” means billion PFUs. As used herein “PP” means plaque-purified. Thus, for example PPMK107 means plaque-purified Newcastle Disease virus strain MK107. As used herein “PFU/m²”, which is a standard unit for expressing dosages, means PFUs per square meter of patient surface area. As used herein the term “replication-competent” virus refers to a virus that produces infectious progeny in cancer cells.

In accordance with this invention the time from the first desensitization dose to the first escalated dose is measured from the end of administration of the first desensitization dose to the beginning of administration of the first escalated dose. In an embodiment of this invention, the first escalated dose is administered from 24 to 36 hours after the first desensitization dose.

In an embodiment of the method of this invention, the one or more desensitization doses are about 2.4×10¹⁰ PFU per square meter of patient surface area, and the one or more escalated doses are about 4.8×10¹⁰ PFU per square meter of patient surface area.

In accordance with the methods of this invention the therapeutic Newcastle Disease Virus utilized can be of low (lentogenic), moderate (mesogenic) or high (velogenic) virulence. The level of virulence is determined in accordance with the Mean Death Time in Eggs (MDT) test. (Alexander, “Chapter 27: Newcastle Disease” in Laboratory Manual for the Isolation and Identification of Avian Pathogens, 3^{rd} ed., Purchase, et al. eds. (Kendall/Hunt, Iowa), page 117.) Viruses are classified by the MDT test as lentogenic (T>90 hours); mesogenic (MDT from 60-90 hours); and velogenic (MDT<60 hours).

In accordance with this invention, any conventional route or technique for administering viruses to a subject can be utilized. In one embodiment of this invention, the virus is administered systemically, for example intravenously. For intravenous administration of a therapeutic virus in accordance with this invention, preferably the virus is a mesogenic strain of Newcastle Disease Virus.

It has been found that undesired side effects can be decreased by controlling the rate at which the virus is administered. When administering a mesogenic strain of Newcastle Disease Virus by the intravenous route, is preferable for a dose of the virus to be administered over an administration time period of up to 24 hours; and the dose to be administered at a rate of up to 7.0×10⁸ PFU per square meter of patient surface area in any ten minute sampling time period within the administration time period. More preferably, the rate at which the dose is administered is up to 2.0×10⁸ PFU per square meter of patient surface area in any ten minute sampling time period within the administration time period. Generally it is convenient to select the rate of administration so that the administration time period is at least 1 hour. Still fewer side effects are generally observed when the administration time period is at least 3 hours. It is especially helpful to control the rate at which the first desensitization dose of the virus is administered.

The subject that is treated in accordance with this invention can be either a human subject or a non-human mammalian subject.

Although monitoring the treatment is not an essential aspect of the invention, there are techniques for measuring the therapeutic effects of the treatment. These include, measuring the size of the tumor after administration of the virus, and a decrease in tumor size is a positive result.

The invention will be better understood by reference to the following examples, which illustrate but do not limit the invention described herein. In the following examples the NDV used was a triple-plaque purified attenuated (mesogenic) version of the MK107 strain of Newcastle Disease Virus, described more fully in International Patent Publication WO 00/62735, published Oct. 26, 2000 (Pro-Virus, Inc.). The entire content of WO 00/62735 is hereby incorporated herein by reference.

### EXAMPLES

### Example 1

### Use of a Desensitizing Dose of PPMK107 to Reduce the Lethality of a Subsequent Dose of PPMK107 given 24 or 48 Hours Later

C3H/Hen mice (9 weeks old) were injected intravenously (over 30 seconds) on day 0 with either vehicle (5% mannitol/1% lysine) or PPMK107 (3E+08 PFU/mouse). A second injection consisting of a PPMK107 dose of 1E+10 PFU/mouse (over 30 seconds) was given at various times later (3 hours, 12 hours, 24 hours and 48 hours). A control set of mice received the first PPMK107 dose of 3E+08 PFU/mouse only with no additional injections. As shown in Table 1 below, almost all mice receiving a first treatment of vehicle died subsequently from the 1E+10 PFU dose (Groups 5 to 8 in Table 1). In contrast, mice receiving 3E+08 PFU of PPMK107 at times 24 and 48 hours before the subsequent higher dose of 1E+10 PFU were protected from lethality (Groups 3 and 4 in Table 1). Giving the desensitizing dose 3 hours or 12 hours before the 1E+10 PFU dose did not block lethality (Groups 1 and 2 in Table 1). These data indicate that PPMK107 can be used to desensitize the lethality of subsequent doses of this same agent when given 24 or 48 hours apart.

TABLE 1
Use of a Desensitizing Dose of PPMK107 to Reduce the Lethality
of a Subsequent Dose of PPMK107 given 24 or 48 hours later.

| | N (Num- | | | | |
|---|---|---|---|---|---|
| | ber of | | Time | | % |
| Group | mice per | Injection on | for 2^{nd} | 2^{nd} | Lethal- |
| # | group) | Day 0, Hour 0 | Injection | Injection | ity |
| 1 | 8 | PPMK107, | Hour 3 | PPMK107, | 88% |
| | | 3E+08 PFU | | 1E+10 PFU | |
| 2 | 8 | PPMK107, | Hour 12 | PPMK107, | 100% |
| | | 3E+08 PFU | | 1E+10 PFU | |
| 3 | 8 | PPMK107, | Hour 24 | PPMK107, | 0% |
| | | 3E+08 PFU | | 1E+10 PFU | |
| 4 | 8 | PPMK107, | Hour 48 | PPMK107, | 0% |
| | | 3E+08 PFU | | 1E+10 PFU | |
| 5 | 8 | Vehicle | Hour 3 | PPMK107, | 88% |
| | | | | 1E+10 PFU | |
| 6 | 8 | Vehicle | Hour 12 | PPMK107, | 100% |
| | | | | 1E+10 PFU | |
| 7 | 8 | Vehicle | Hour 24 | PPMK107, | 100% |
| | | | | 1E+10 PFU | |
| 8 | 8 | Vehicle | Hour 48 | PPMK107, | 100% |
| | | | | 1E+10 PFU | |
| 9 | 6 | PPMK107, | No 2^{nd} | No 2^{nd} | 0% |
| | | 3E+08 PFU | Injection | Injection | |

What is claimed is:

1. A method for treating a mammalian subject having a tumor, comprising administering to the subject an amount of a Newcastle disease virus effective to treat the subject, wherein
the virus is administered to the subject in one or more cycles;
at least one cycle comprises administering sequentially one or more desensitization doses of the followed by one or more escalated doses of the virus to the subject;
the amount of the virus in each escalated dose is higher than the amount of virus in each desensitization dose; and
the first escalated dose is administered from 18 to 36 hours after the first desensitization dose.

2. The method of claim 1, wherein the first escalated dose is administered from 24 to 36 hours after the first desensitization dose.

3. The method of claim 1, wherein the virus is a mesogenic strain of Newcastle Disease Virus.

4. The method of claim 1, wherein the virus is administered systemically.

5. The method of claim 4, wherein the virus is administered intravenously.

6. The method of claim 5, wherein the virus administered is a mesogenic strain of Newcastle Disease Virus.

7. The method of claim 1, wherein the virus dose is administered over an administration time period of up to 24 hours; and the dose is administered at a rate of up to 7.0×10⁸ PFU per square meter of patient surface area in any ten minute sampling time period within the administration time period.

8. The method of claim 7, wherein the rate is up to 2.0×10⁸ PFU per square meter of patient surface area in any ten minute sampling time period within the administration time period.

9. The method of claim 7, wherein the administration time period is at least 1 hour.

10. The method of claim 9, wherein the administration time period is at least 3 hours.

11. The method of claim 1, wherein the subject is a human subject.

12. The method of claim 1, wherein the subject is a non-human mammal.

13. The method of claim 1, wherein the size of the tumor decreases after administration of the virus.
